(19) United States
(12) Patent Application Publication
(10) Pub. No.: US 2016/0215054 A1
(43) Pub. Date: Jul. 28, 2016

(54) TREATMENT OF DIABETES MELLITUS, METHODS AND COMPOSITIONS THEREFOR

(71) Applicants: Emil R. Unanue, St. Louis, MO (US); Kenneth M. Murphy, St. Louis, MO (US); Javier A. Carrero, St. Louis, MO (US)

(72) Inventors: Emil R. Unanue, St. Louis, MO (US); Kenneth M. Murphy, St. Louis, MO (US); Javier A. Carrero, St. Louis, MO (US)

(73) Assignee: Washington University, St. Louis, MO (US)

(21) Appl. No.: 15/005,542

(22) Filed: Jan. 25, 2016

Related U.S. Application Data
(60) Provisional application No. 62/107,061, filed on Jan. 23, 2015.

(51) Int. Cl. C07K 16/28; C07K 16/24; C07K 16/26

(52) U.S. Cl. CPC C07K 16/2866; C07K 16/26; C07K 16/2833; C07K 16/24; C07K 2317/76

(57) ABSTRACT

Methods of treatment of diabetes are disclosed. These methods include administration of an inhibitor of chemokine XCL1, and administration of an inhibitor of XCL1 receptor XCR1. Inhibition of formation of CD103⁺ dendritic cells, inhibition of their migration into islets, or inhibition of function of CD103⁺ dendritic cells can be used to prevent, treat or manage diabetes.

### CROSS REFERENCE TO RELATED APPLICATIONS

This application claims the benefit of U.S. Provisional Application No. 62/107,061, filed Jan. 23, 2015, the disclosure of which is hereby incorporated by reference in its entirety.

### GOVERNMENTAL RIGHTS

This work was funded by National Institutes of Health Grants DK058177 and DK020579 and the National Institute of Arthritis and Musculoskeletal and Skin Diseases grant P30AR048335. The Government of the United States has certain rights in the invention.

### FIELD OF THE INVENTION

The present disclosure provides methods of preventing, treating and managing diabetes that can provide alternatives or supplements to insulin administration.

### BACKGROUND OF THE INVENTION

Diabetes mellitus is a chronic metabolic disease that is becoming increasingly prevalent in the population. Diabetes is characterized by inability of the body to properly regulate blood glucose either due to defects in insulin production or development of insulin resistance in a subject. Either of these defects will eventually lead to chronic hyperglycemia.

There are two types of diabetes: Type I diabetes, which is an early onset disease caused mostly by genetic factors, and Type II diabetes, which is a late onset disease strongly linked to obesity. Type II diabetes is characterized by cellular stressors from insulin resistance in a subject leading to insulin overproduction that weakens the insulin producing β-cells in the Islets of Langerhans (Sun AC et al; World J Diabetes, 2014 December 15; 5(6):739-746). Similarly, Type I diabetes is caused by failure of β-cells to produce insulin. However, in contrast to Type II diabetes, this failure can be caused by autoreactive T-cells that target β-cells for destruction by immune-mediated pathways (Sun AC et al, supra).

Treatment of diabetes by insulin administration is well-established. However, insulin therapy has associated problems, such as requirements for aggressive daily treatment, including blood glucose monitoring and twice daily insulin injections, or the use of an insulin pump (Soni A and Ng SM World J Diabetes, 2014 Dec. 15; 5(6)877-881). These regimens require strict patient compliance, which can often require a degree of self-discipline not always possessed by human diabetic subjects. Furthermore, incorrect dosage or timing of an insulin injection can have serious medical consequences.

### SUMMARY OF THE INVENTION

In view of the problems associated with insulin therapy for diabetes, the present inventors have developed methods of preventing, treating and managing diabetes that can provide alternatives or supplements to insulin administration.

The present inventors have discovered that the development of diabetes in non-obese diabetic (NOD) mice, a model system, can be prevented in genetic “knock-out” mice by eliminating function of the gene for transcription factor Batf3. The present inventors also discovered that islets of Langerhans comprise CD103⁺ dendritic cells. The present inventors further discovered that genetic knockouts of NOD mice that lack transcription factor Batf3 also lack CD103⁺ dendritic cells in the islets of Langerhans. Furthermore, such mice do not develop diabetes. At 3-4 weeks of age in NOD mice, three events take place: (1) an increase in the CD103⁺ DCs, (2) the localization of CD4⁺ T cells inside islets, presumably insulin reactive, and (3) changes in gene expression. Notably, the installment of the diabetic process requires two interconnected cells: the presence of the CD103⁺ DCs and of CD4⁺ T cells that recognize immunogenic insulin. The absence of the CD103⁺ DC lineage in the NOD.Batf3^{-/-} mouse results in a quiescent islet preventing the autoreactive diabetic process for the life of the mouse. In addition, the present inventors have identified chemokine XCL1 (lymphotactin) as a chemotactic attractant for CD103⁺ dendritic cells of the islets, as well as the presence of cell-surface receptors for XCL1 in the CD103⁺ dendritic cells of the islets, i.e., the G protein-coupled receptor XCR1. The inventors have determined that the CD103⁺ dendritic cells of the islets control the initiation of diabetes; subjects without CD103⁺ dendritic cells do not develop diabetes. Inhibition of formation of CD103⁺ dendritic cells, inhibition of their migration into islets, or inhibition of function of CD103⁺ dendritic cells, can provide means for preventing, treating or managing diabetes.

In various embodiments, methods of diabetes prevention, treatment or management of the present teachings comprise administering to a subject in need thereof a therapeutically effective amount of an antagonist of the class C chemokine XCL1. In some configurations, the XCL1 antagonist can be a neutralizing antibody against XCL1, or an XCL1-neutralizing fragment thereof, such as, for example, an Fab fragment. In various aspects, an anti-XCL1 antibody can be a polyclonal antibody or a monoclonal antibody. In various aspects, an anti-XCL1 antibody can be a mouse monoclonal antibody, a hamster monoclonal antibody, a single chain camelid antibody or a humanized monoclonal antibody. In various aspects, the antibody binds to, or can neutralize activity of, human XCL1. In various configurations, the antibody can be directed against human XCL1, or can be directed against a non-human XCL1 such as mouse XCL1, but can be cross-reactive against human XCL1. In some configurations, the XCL1 antagonist can inhibit migration of CD103⁺ dendritic cells (DCs) into islets of Langerhans.

In various embodiments, methods of diabetes prevention, treatment or management of the present teachings comprise administering to a subject in need thereof a therapeutically effective amount of an antagonist of XCR1, i.e., the cell surface receptor for class C chemokine XCL1 (lymphotactin). In some configurations, the XCR1 antagonist can be a neutralizing antibody against XCR1 (e.g., US Patent Application Publication 2014/0193421 A1 of Sakamoto et al.), or an XCR1-neutralizing fragment thereof, such as, for example, an Fab fragment. In various aspects, an anti-XCR1 antibody can be a polyclonal antibody or a monoclonal antibody. In various aspects, an anti-XCR1 antibody can be a mouse monoclonal antibody, a hamster monoclonal antibody, a single chain camelid antibody or a humanized monoclonal antibody. In various aspects, the antibody binds to, or can neutralize activity of, human XCR1. In various configurations, the antibody can be directed against human XCR1, or can be directed against a non-human XCR1 such as mouse XCR1, and can be cross-reactive against human XCR1. In some configurations, the XCR1 antagonist can inhibit migration of CD103+ dendritic cells (DCs) into islets of Langerhans.

In various embodiments, methods of diabetes prevention, treatment or management of the present teachings can comprise administering to a subject in need thereof a therapeutically effective amount of a combination of antagonists of XCL1 and XCR1, In some configurations, the antagonists can be neutralizing antibodies against XCL1 and XCR1, or neutralizing fragments thereof, such as, for example, Fab fragments. In various aspects, the antibodies can be polyclonal antibodies and/or a monoclonal antibodies. In various aspects, the antibodies can be any combination of mouse monoclonal antibody, a hamster monoclonal antibody, single chain camelid antibody and/or humanized monoclonal antibody. In various aspects, the antibodies can bind to or can neutralize activity, of human XCL1 and XCR1. In various configurations, the antibodies can be directed against human XCL1 and XCR1, or can be directed against a non-human XCL1 and XCR1 such as mouse XCL1 and XCR1, but can be cross-reactive against human XCL1 and XCR1. In some configurations, the XCL1 and XCR1 antagonists can inhibit migration of CD103⁺ dendritic cells (DCs) into islets of Langerhans.

In some embodiments, methods of diabetes treatment, prevention or management can comprise administering to a subject in need thereof an effective amount of an antibody, or an antigen-binding fragment thereof, against an insulin peptide-MHC complex. In some configurations, the insulin peptide-MHC complex can be an insulin peptide-MHC class II complex. In some configurations, the antibody can be directed against an epitope defined by an insulin peptide bound to the binding groove of an MHC class II molecule. In some configurations, the insulin peptide-MHC complex can be comprised by an antigen presenting cell (APC) such as a macrophage or a dendritic cell, which can present the complex to a T cell such as a CD4⁺ T cell. In some configurations, the insulin peptide-MHC complex can be presented by a CD103⁺ dendritic cell. In some configurations, the CD103⁺ dendritic cell which presents the complex can be comprised by an islet of Langerhans. In various aspects, an anti-insulin peptide-MHC complex antibody can be a mouse monoclonal antibody, a hamster monoclonal antibody, a single chain camelid antibody or a humanized monoclonal antibody, or an epitope-binding fragment thereof. In various configurations, an insulin peptide-MHC complex of the present teachings can be used to neutralize CD4⁺ T cells that initiate diabetogenesis.

In various embodiments, methods of diabetes prevention or treatment of the present teachings can comprise administering to a subject in need thereof a therapeutically effective amount of an inhibitor of chemotactic migration of CD103⁺ dendritic cells, such as, without limitation, an inhibitor of G-protein binding to G-protein-coupled receptor, such as, for example, pertussis toxin. In various configurations, the inhibitor can interfere with chemotactic migration of CD103⁺ dendritic cells into islets of Langerhans.

In some embodiments, a mouse can comprise a mutation in the XCR1 gene. In some configurations, the mutation can be a loss of function mutation. In some configurations, the mutation can be an insertion that disrupts expression of XCR1 protein.

In some embodiments, NOD.Batf^{-/-} mice can be used as a model system for diabetes research. In some configurations, mice can comprise NOD mice that have been backcrossed to an allele of Batf^{-/-} until all the microsatellite markers for insulin dependent alleles are present. In various aspects, these mice can then be used for research into the development of dendritic cells and the role of dendritic cells in various autoimmune diseases. In some embodiments, NOD.Batf^{-/-} mice can be used in drug development assays.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A depicts the incidence of diabetes in relevant phenotypes. While Nod.Batf3^{+/-} showed diabetic progression equivalent to that of NOD mice, NOD.Batf^{-/-} mice did not develop diabetes. FIG. 1B, FIG. 1C, FIG. 1D depict the morphology of mouse islets. While NOD mouse islets have intraislet insulitis and peri-insulitis (FIG. 1B, representative 6 week old female), NOD.Batf3^{-/-} pancreata have normal morphology (FIG. 1C and FIG. 1D, 6 week and 52 week female, respectively). FIG. 1E, FIG. 1F, FIG. 1G, FIG. 1H, FIG. 1I depict the detection of immune system cells in mice. Flow cytometry studies of mouse antigen presenting cells in NOD and NOD.Batf3^{-/-} mice revealed reductions in CD8α cells in the thymocytes (FIG. 1E), splenocytes (FIG. 1F) and spleen (FIG. 1I). Reduction in CD103⁺ was observed in the pancreatic lymph nodes (pLN) (FIG. 1G) and mesenteric lymph nodes (FIG. 1H) in NOD.Batf3^{-/-} mice relative to NOD mice.

### DETAILED DESCRIPTION OF THE INVENTION

The present teachings include descriptions that are not intended to limit the scope of any aspect or claim. Unless specifically presented in the past tense, an example can be a prophetic or an actual example. The examples and methods are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

Methods and compositions described herein utilize laboratory techniques well known to skilled artisans. Such techniques can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; Behringer, R., et al., Manipulating the Mouse Embryo: A Laboratory Manual, Fourth Edition, Cold Spring Harbor Laboratory Press, Cold Spring Harbor, N.Y., 2014; Harlow, E., Using Antibodies: A Laboratory Manual, Cold Spring Harbor Laboratory Press, Cold Spring Harbor, N.Y., 1999; Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

All publications cited herein are incorporated by reference, each in its entirety. As used herein, the singular forms “a”, “an” and “the” are intended to include the plural forms as well, unless the context indicates otherwise.

Antibodies can be created through several standard methods set forth in many standard laboratory manuals. Hamster antibodies can be produced using the method of R. D. Schreiber published in Uppaluri et al.; Transplantation 2008 Jul. 15; 86(1):137-147 and Sheehan et al.; J Immunol. 1988 140:4231-4237, each of which is hereby incorporated by reference, each in its entirety. Briefly, Armenian hamsters are immunized with peptide in adjuvant, and then boosted with a second dose of peptide several weeks later. Immunity can be screened using standard ELISA methods. Hybridomas can be generated and supernatants can be screened for immunoreactivity.

Mutant mice can be created using any standard method of mutagenesis known to persons skilled in the art.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A physician diagnoses a patient in the early stages of type I diabetes. The physician administers an anti-XCL1 monoclonal antibody. This treatment prevents further degradation of the islets of Langerhans. The patient never requires insulin.

### Example 2

A physician diagnoses a patient in the early stages of type I diabetes. The physician administers an anti-XCR1 neutralizing antibody, and prevents further degradation of the patient's islets of Langerhans. The patient never requires insulin.

### Example 3

A physician diagnoses a patient in the early stages of type I diabetes. The physician administers an XCR1 antagonist which selectively blocks signal transduction between XCR1 and a G protein. Further degradation of the islets of Langerhans is halted. The patient never requires insulin.

### Example 4

A physician diagnoses a patient in the early stages of type I diabetes. The physician administers an inhibitor which selectively blocks binding between XCL1 and XCR1. Further degradation of the islets of Langerhans is prevented. The patient never requires insulin.

### Example 5

A physician diagnoses a patient in the early stages of type I diabetes. The physician prescribes an effective amount of an insulin peptide-MHC complex neutralizing antibody. Further degradation of the islets of Langerhans is halted, and the patient never requires insulin.

### Example 6

This example illustrates experiments implicating XCR1 and XCL1 in the lymphocyte infiltration of the islets of Langerhans.

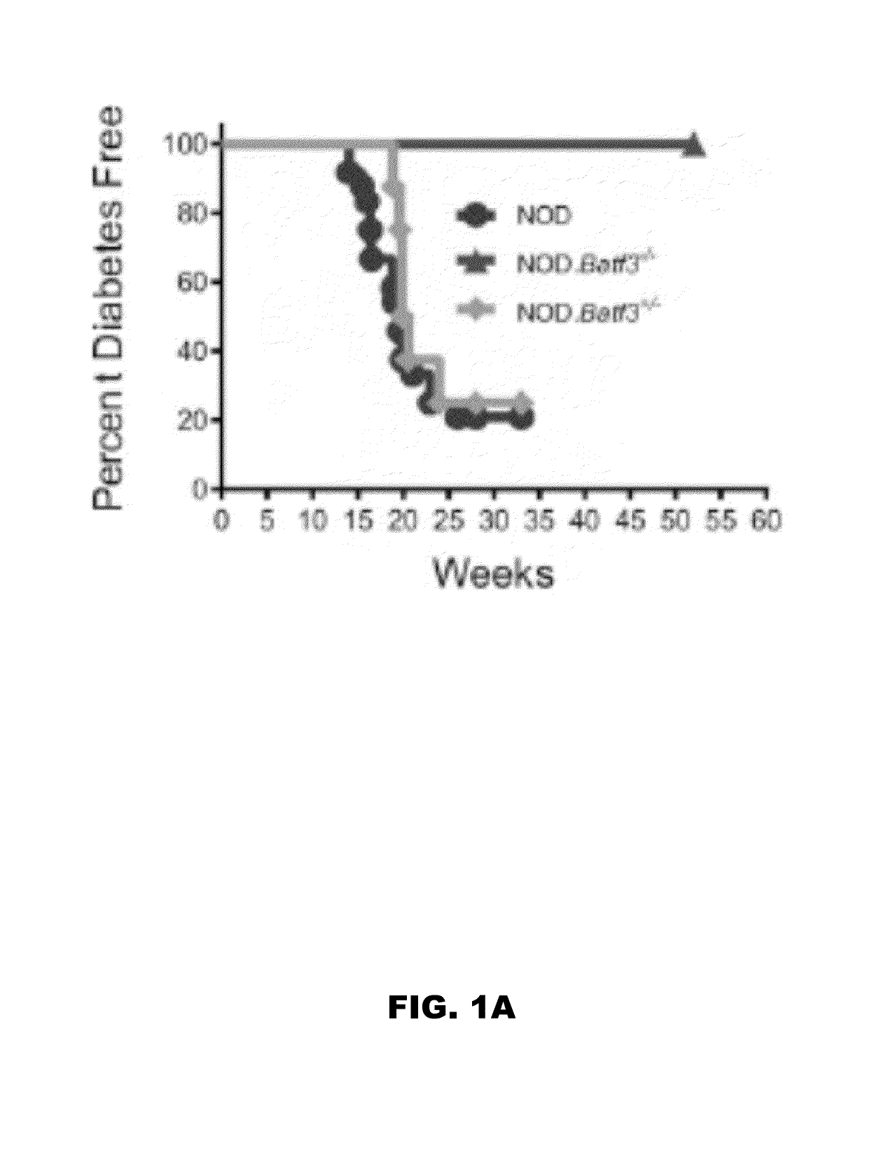

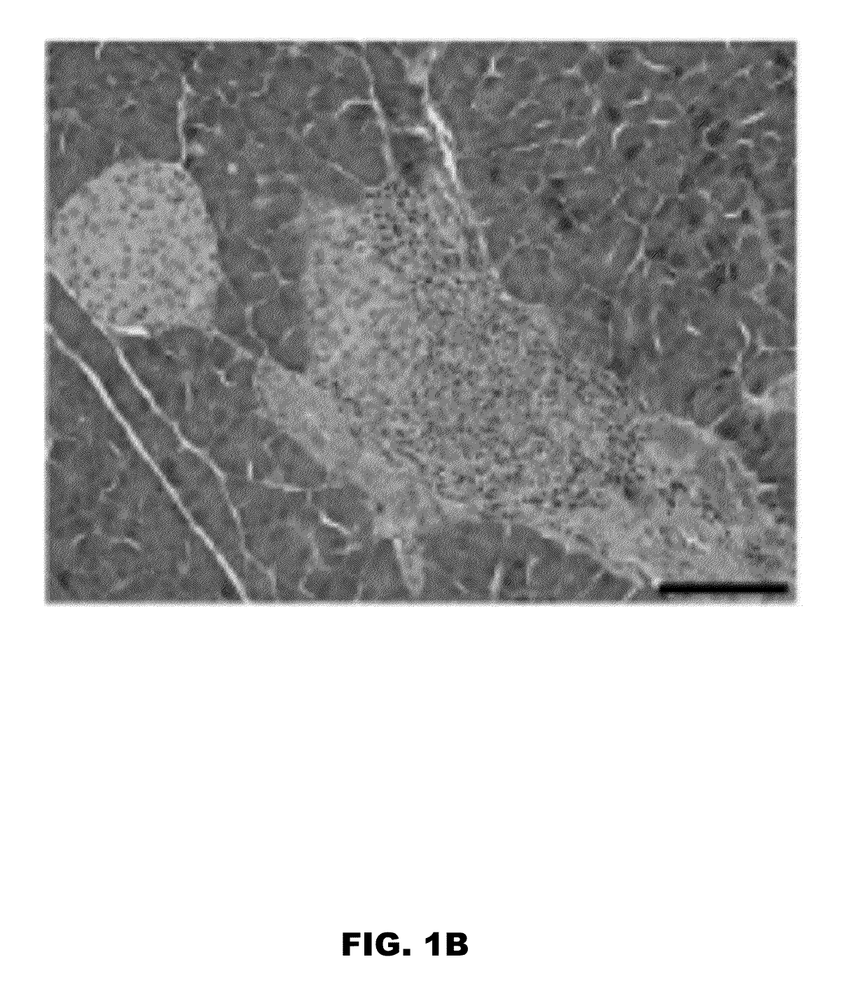

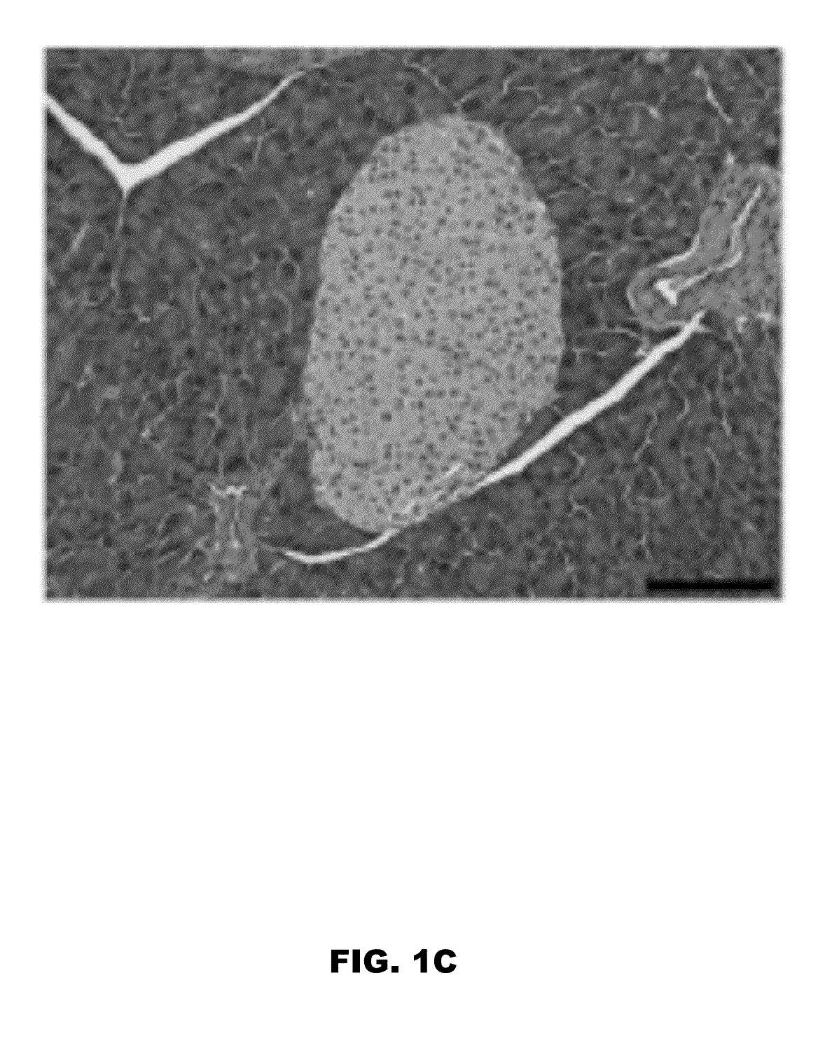

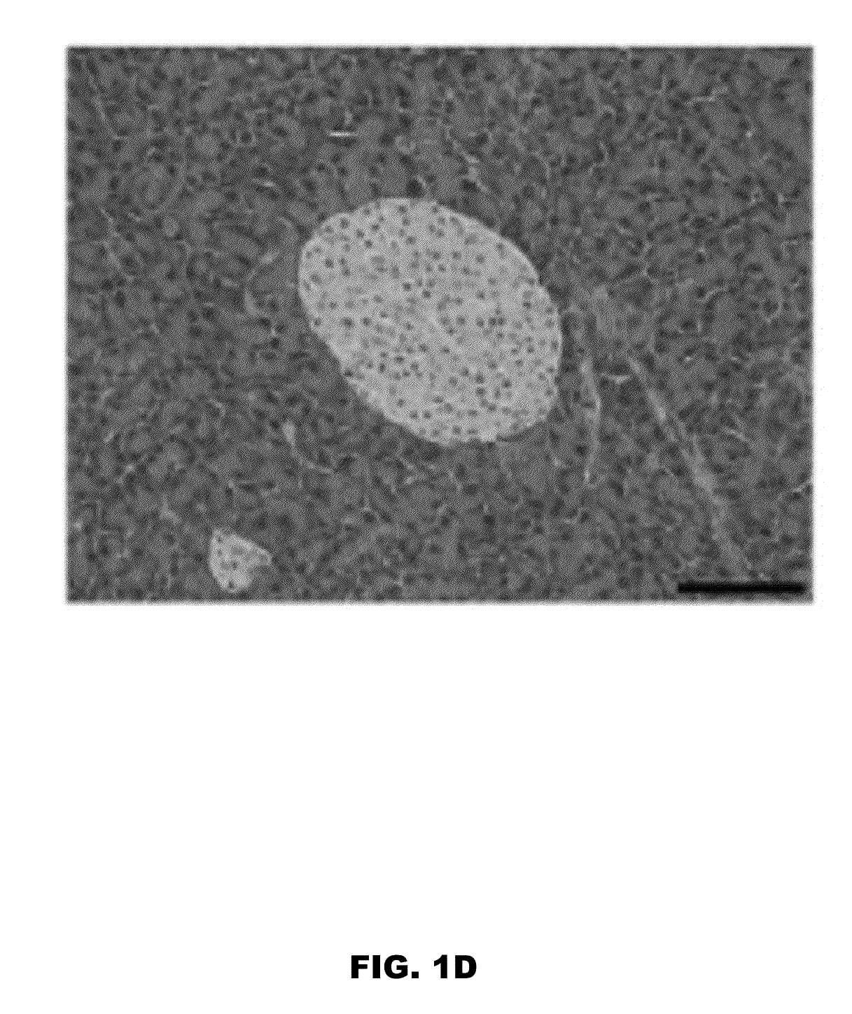

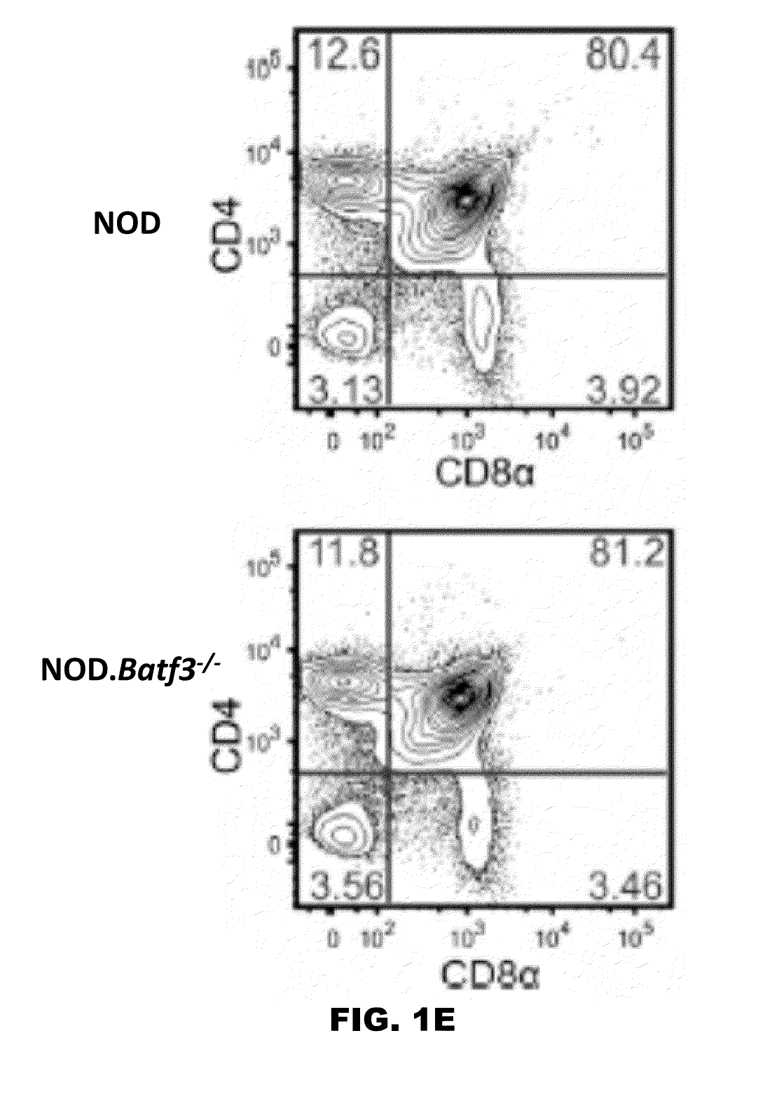

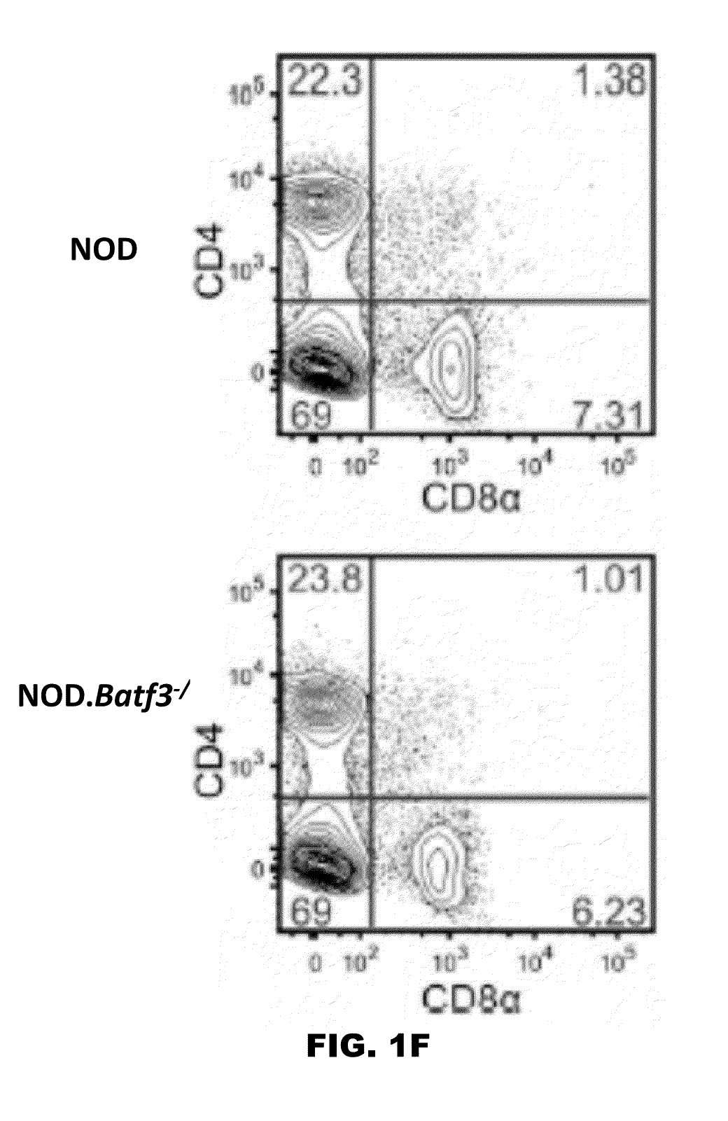

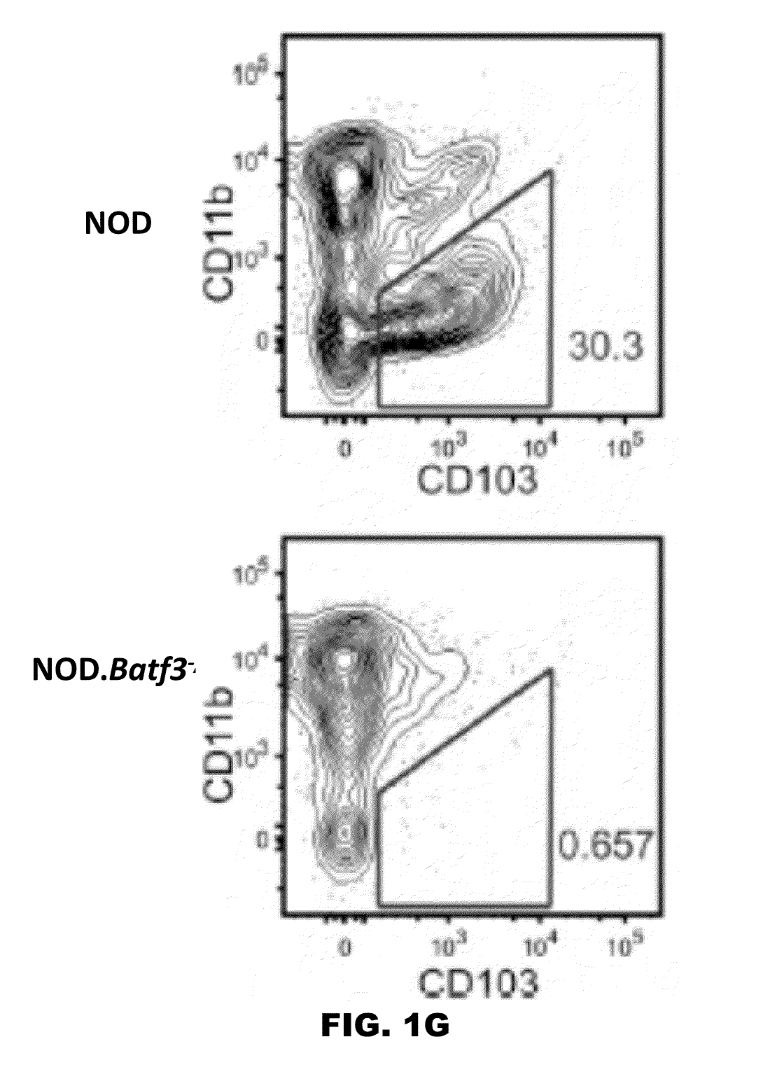

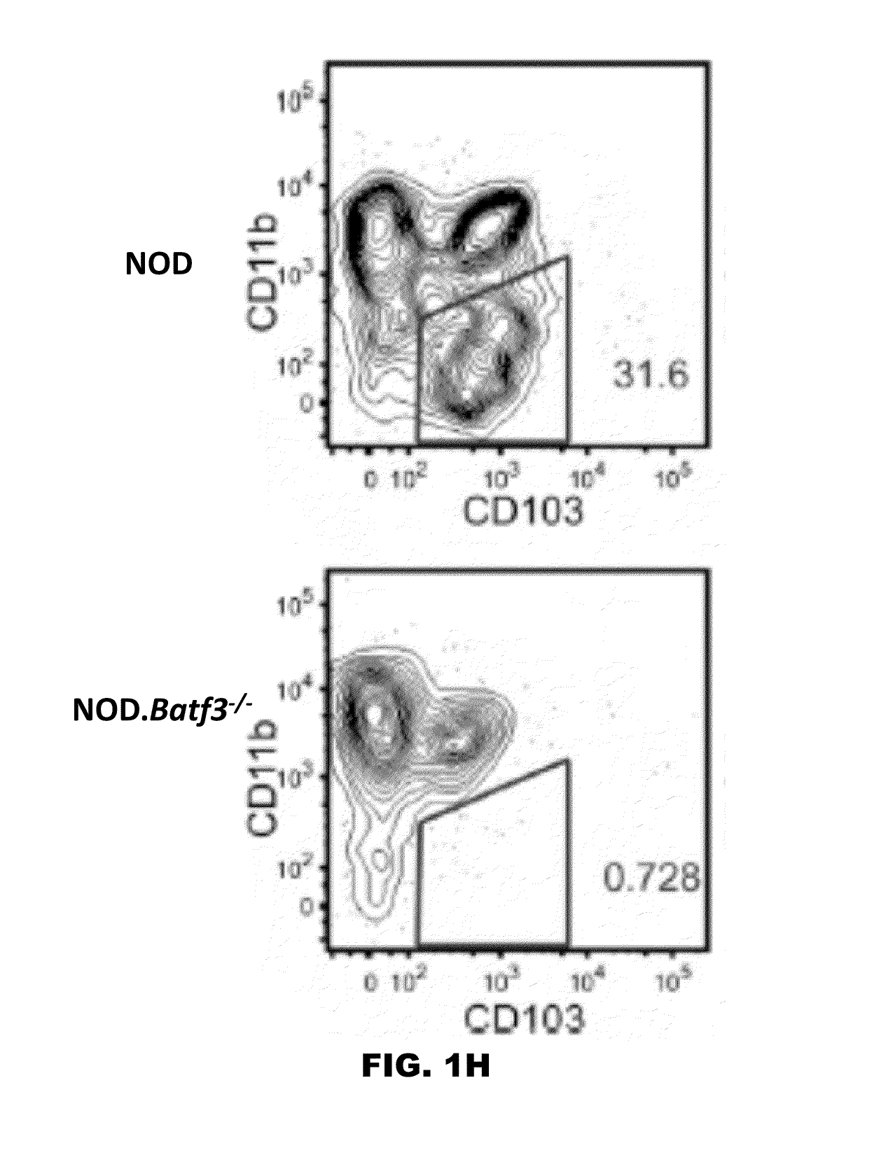

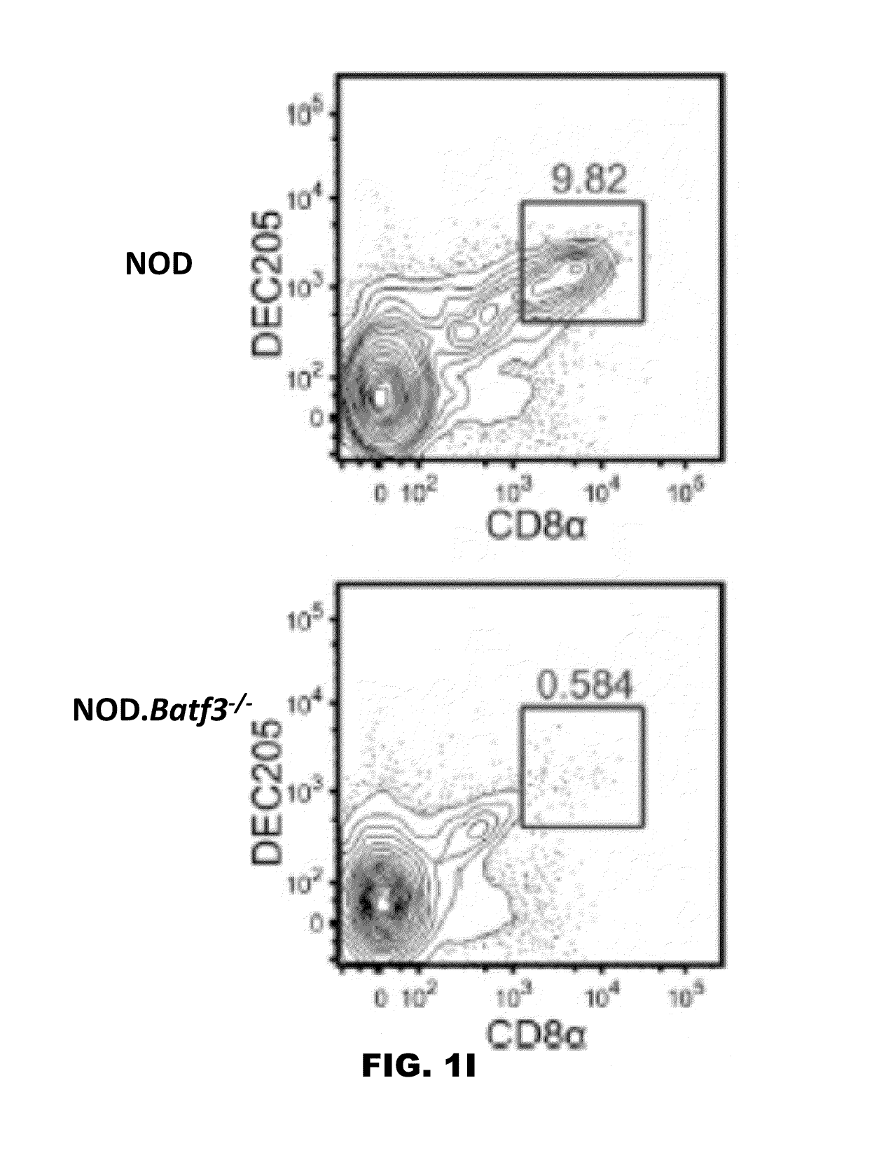

In these experiments, NOD mice were crossed into a knockout line of Batf, a transcription factor that, among other things, regulates the differentiation of CD103⁺ dendritic cells. The present inventors back-crossed 129S6.Batf3^{-/-} mice (Hilder et al.; Science; 2008; 322: 1097-1100) to NOD mice and performed single nucleotide polymorphism analyses to preserve insulin-dependent susceptibility alleles (Ferris et al.; Immunity; 2014 Oct. 16; 41: 657-669). While Nod.Batf3^{+/-} showed diabetic progression equivalent to that of NOD mice, NOD.Batf^{-/-} mice did not develop diabetes (FIG. 1A). In accordance with this finding, the inventors observed that while NOD mouse islets have intraislet insulitis and peri-insulitis (FIG. 1B, representative 6 week old female), NOD.Batf3^{-/-} pancreata have normal morphology (FIG. 1C and FIG. 10, 6 week and 52 week female, respectively.) Flow cytometry studies of mouse antigen presenting cells revealed reductions in CD8α cells in the thymocytes (FIG. 1E), splenocytes (FIG. 1F) and spleen (FIG. 1I). Reduction in CD103⁺ was observed in the pancreatic lymph nodes (pLN) and mesenteric lymph nodes (FIG. 1G and FIG. 1H, respectively). These data illustrate that CD103 and CD8α cells are not present in NOD.Batf3^{-/-} mice. Because these mice do not develop diabetes, these cell types are required for diabetic autoimmunity.

What is claimed is:

1. A method of diabetes prevention, treatment or management, the method comprising administering to a subject in need thereof a therapeutically effective amount of an antagonist of the class C chemokine XCL1.

2. The method of claim 1, further comprising administering a therapeutically effective amount of an antagonist of the cell surface receptor for class C chemokine XCL1 (lymphotactin; XCR1).

3. A method of diabetes prevention, treatment or management, the method comprising administering to a subject in need thereof a therapeutically effective amount of an antagonist of the cell surface receptor for class C chemokine XCL1 (lymphotactin; XCR1).

4. The method of claim 3, further comprising administering a therapeutically effective amount of an antagonist of the class C chemokine XCL1.

5. A method of diabetes prevention or treatment, the method comprising administering to a subject in need thereof a therapeutically effective amount of an inhibitor of chemotactic migration of CD103⁺ dendritic cells.
